(19) United States
(12) Patent Application Publication
(10) Pub. No.: US 2012/0240431 A1
(43) Pub. Date: Sep. 27, 2012

(54) Turf Toe Terminator (TTT)

(76) Inventor: Christopher Colclough, (US)

(21) Appl. No.: 13/053,895

(22) Filed: Mar. 22, 2011

(51) Int. Cl. A61F 5/14

(52) U.S. Cl. 36/140

(57) ABSTRACT

A turf toe terminator is an insole which is made up of a semi flexible orthotic insole which is inserted into the cleat or sneaker or is created as part of the sneaker or cleat to help prevent injury due to hyperextension of the great toe (turf toe).

The present invention is directed to an apparatus for helping to prevent exacerbation of injury due to hyper-extension of the metatarso phalangeal joint of the great toe (turf toe), especially by athletes, and to alleviating pain so that the person may continue to function somewhat normally after such an injury and experience less discomfort until the injury has been rehabilitated. A turf toe terminator is an insole insert or can be made as part of the cleat or sneaker sole. Either way, the TTT is made up of a semi flexible orthotic insole which is inserted into or made part of a cleat or sneaker. If it is an insert, it should replace the current insole. The apparatus is created to allow for a natural extension of the great toe. The orthotic will have a nylon strap that will run from the tip of the toe to the heal of the orthotic. The nylon strap will be connected at both ends of the orthotic and generate a natural arch of the orthotic. The strap together with the orthotic will allow the great toe to have dorsal Extension, while preventing the toe from hyperextension. This will help prevent injury and provide support to an already hyperextension of the great toe, especially during work or athletic events.

### BACKGROUND OF THE INVENTION

The present invention is directed to an apparatus for helping to prevent exacerbation of injury due to hyper-extension of the metatarso phalangeal joint of the great toe, especially by athletes, and alleviating pain so that the person may continue to function somewhat normally after such an injury and experience less discomfort until the injury has rehabilitated.

In many athletic events and in some types of non-athletic occupations, the first metatarsophalangeal joint (MP joint) becomes injured because the great toe is suddenly hyper-extended. A sudden hyperextension of the great toe can vary in degree, the joint capsule can be torn from the metatarsal head, articular cartilage damage can occur, or sometimes a fracture of the bone structure can occur. This injury is commonly referred to as “Turf-toe” because of its increased regularity since the introduction of artificial turf Turf toe injuries are common in many types of athletic events such as football, soccer, lacrosse and basketball, especially when the athlete is forced toward the ground with the heel in the air causing the MP joint to take all of weight.

A sprain of the MP joint can be debilitating in that the great toe is very important in weight bearing, and the proximal joint must bear the brunt of every step. Turf-toe injuries can be extremely painful and typically require at least six weeks to rehabilitate. During the rehabilitation period, an individual may be in such severe pain from the injury whenever the toe extends upward making it difficult for the individual to walk on the foot, even for normal use. In the absence of a device for restricting hyperextension, an athlete, who applies greater flexure to the toe during sporting events, is essentially unable to perform during the rehabilitation period.

I have devised an orthotic insole that when utilized will allow the great toe to move in a dorsal extension position utilizing the orthotic constructed out of a combination of an insole made out of polypropylene and a nylon strap. Full extension occurs when the toe extends upward to the shin. By allowing the great toe to extend to a fully extended position and not allowing it to hyperextend, less stress is placed upon the great toe and the stress is actually diverted to the heel of the shoe pushing down towards the ground. This ensures that there will be less discomfort, less chance or injury and enable the athlete to continue to perform at a high level.

### SUMMARY OF THE INVENTION

A turf toe insole or sole in accordance with the invention includes five components. The first is a rigid polypropylene plastic insole. The second is a nylon strap that is attached to the bottom of the plastic insole using nylon string (third component). It is attached to the toe and the heal of the plastic insole. The nylon strap will be attached in such a way that it will have as much tension to generate an inverted arch in the orthotic. This will help prevent and provide support to an already hyper-extension of the great toe, especially during workouts or athletic events. The fourth and fifth components are latex foam insoles that are attached to one another and then attached to the plastic orthotic. It is attached using the sixth and final component; rubber cement

In either application, it is required that a high top sneaker or cleat is utilized.

During activity, when the toes forced upward (dorsal extension) the orthotic along with the nylon strap will not allow the toes to extend to hyperextension and will transfer the pressure from the toes to the heel of the shoe pushing downward.

### DESCRIPTION

### Objects and Advantages of the Invention

Therefore, the object of the present invention are to provide a device that substantially maintains a great toe normal motion such that the supporting structures of the MP joint associated with the toe will not be hyper extended during walking, running, sporting events, working. This support will not only prevent injury but it will allow the user to continue to be active during the rehabilitation of the injury; to provide such a device which is extremely easy to insert or can easily be created as part of the sneaker or cleat; such a device which is relatively low profile so as to be comfortable in use and which allows application of other support to the user's foot such as ankle taping; to provide such a device which is highly effective in alleviating pain associated with turf toe so that the user can function with a large degree of normalcy; easy to use and particularly adapted for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a side view of the turf toe terminator.

FIG. 2 is a side view of the orthotic shown with a great toe extension.

FIG. 2 is a bottom view and a lateral view of TTT

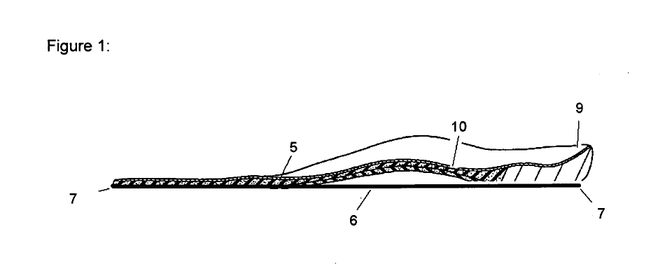

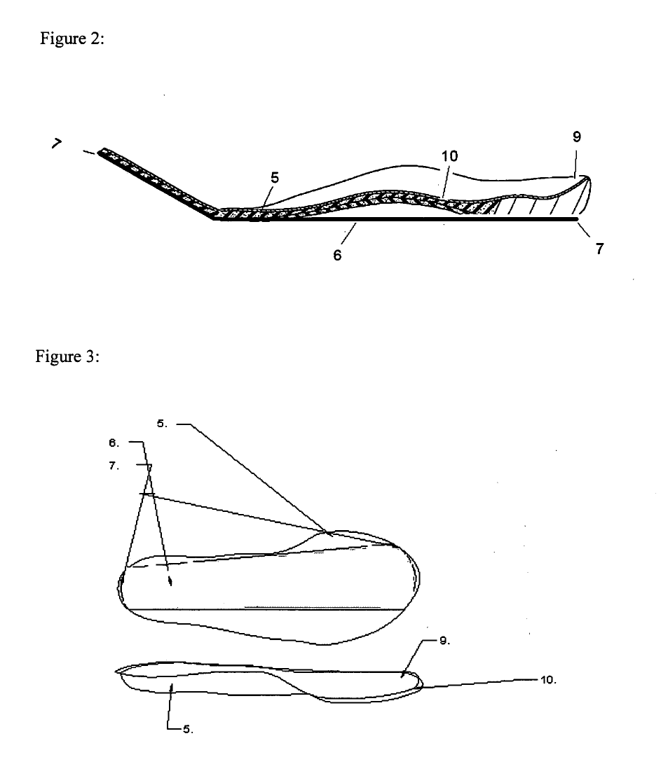

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The reference numeral 1 generally represents a turf toe terminator in accordance with the present invention. The TTT1 includes a Polypropylene insole orthotic 5, a nylon strap 6, Nylon Thread 7, rubber insole 9, adhesive 10.

The orthotic (5) replaces the insole is created as part of a high top Sneaker/cleat or boot, the nylon strap (6) is placed under the orthotic (5), the nylon thread (7) is used to attach the nylon strap (6) to the orthotic (5), the strap (6) will be secured to the toe and the heal of the orthotic (5), the rubber insole (9) will be attached to the top of the orthotic (5) using a rubber adhesive (10).

It is foreseen that other types of anchor mechanisms could be used in conjunction with the invention. For example, threaded screws

It is to be understood that while certain forms of the present invention have been illustrated and described herein, it is not to be limited to the specific forms or arrangement of parts described and shown.

What is claimed and desired to be secured by Letters Patent is as follows:

1. TTT will reduce the chance of hyper-extension of the great toe: and to avoid further injury to great toe after sustaining a turf toe injury:
a) TTT is a semi flexible orthotic insole. The orthotic is made out of polypropylene. It can be made as an insole insert or as part of the cleat or sneaker.
b) TTT will have a nylon strap that will be placed under the orthotic and it will run from the tip of the toe to the heal of the orthotic. The nylon strap will be attached to the orthotic using polyester string. The nylon strap will be attached in such a way that it will have as much tension to generate an inverted arch in the orthotic. This will help prevent and provide support to an already hyper-extension of the great toe, especially during workouts or athletic events;
c) A high-top shoe will be required with this orthotic to avoid heal slippage.
d) The orthotic with the nylon strap will provide the Great Toe with a natural motion but will not allow the toe to hyper extend

2. The orthotic according to claim 1 wherein:
a) Said insole is a semi flexible polypropylene insole insert placed in the shoe.

3. The insole according to claim 2 wherein:
a) Said insole is semi flexible insole insert is adapted to replace existing insole and fits under the effected foot of the user.
b) Said insole is semi flexible insole is created as part of the high top sneaker or cleat
